(19) United States
(12) Patent Application Publication
(10) Pub. No.: US 2024/0245586 A1
(43) Pub. Date: Jul. 25, 2024

(54) MINERAL OIL-FREE LIPSTICK WITH HIGH GLYCERIN CONTENT

(71) Applicant: BEIERSDORF AG, Hamburg (DE)

(72) Inventors: Jessica RATHSACK, Buxtehude (DE); Tanja HARTWIG, Bad Oldeslohe (DE); Dorothe FIEDLER, Hamburg (DE)

(21) Appl. No.: 18/413,677

(22) Filed: Jan. 16, 2024

(30) Foreign Application Priority Data
Jan. 19, 2023 (DE) 102023200406.2

(51) Int. Cl. A61K 8/06; A61K 8/34; A61Q 1/06

(52) U.S. Cl. CPC A61K 8/06; A61K 8/345; A61Q 1/06; A61K 2800/30; A61K 2800/92

(57) ABSTRACT

A cosmetic lipstick based on a glycerin-in-oil emulsion comprising
(a) Diisostearoyl Polyglyceryl-3 Dimer Dilinoleate,
(b) Polyglyceryl-4 Diisostearate/Polyhydroxystearate/Sebacate,
(c) sunflower wax,
(d) carnauba wax.

The emulsion is free of mineral oil, paraffin wax, microcrystalline wax, shellac wax, polyethylene waxes, polyacrylates and crosslinked acrylate/C10-C30 alkyl acrylate polymers.

### CROSS-REFERENCE TO RELATED APPLICATIONS

The present application claims priority under 35 U.S.C. § 119 of German Patent Application No. 102023200406.2, filed Jan. 19, 2023, the entire disclosure of which is expressly incorporated by reference herein.

### BACKGROUND OF THE INVENTION

### 1. Field of the Invention

The present invention relates to a cosmetic lipstick based on a glycerin-in-oil emulsion as well as the production process thereof.

### 2. Discussion of Background Information

The desire to look beautiful and attractive has been rooted in humans for thousands of years. Although ideals of beauty have changed over time, the pursuit of a flawless appearance has always been aimed for by humans, since a pleasant appearance increases their self-esteem and attractiveness to their fellow human beings.

The term “decorative cosmetics” comes from the Latin “decoratio”—the emphasizing of beauty. In most cases, colorants are used to emphasize individual parts of the body, especially on the face, and to lessen color inconsistencies.

In addition to face powders and rouge as powdered cosmetics and stick-shaped preparations, creamy preparations such as day creams and cream make-up based on emulsions are used for this purpose.

Lipstick is one of the most used decorative cosmetics. More than 50% of women in Germany use it. In addition to accentuating the color of the lips, it usually also serves for lip care.

Lipsticks generally consist of a wax matrix in which low concentrations of liquid and semi-solid oils and also pigments and fillers are incorporated. They are in solid stick form.

A disadvantage of the prior art is the fact that lip care products, such as lip creams, lip butter, etc. and particularly the lipsticks of the prior art, often comprise mineral oils and paraffin waxes, which are increasingly unpopular with consumers. In addition to a general desire for products made from renewable raw materials, a role is also played here by the concern about health-endangering impurities that could be present in the raw materials produced from crude oil. Whether this concern is actually justified can be left open in the context of the present disclosure. The fact is that there is a desire among many consumers for “mineral oil-free” products.

In view of the foregoing, it would be advantageous to have available a “mineral oil-free” lipstick.

In order to improve the care properties of lipsticks, glycerin is often added to the formulations. Glycerin serves to moisturize the lips and ensures that the lips do not dry out and become cracked.

However, when producing mineral oil-free or paraffin wax-free lipsticks with relatively high glycerin contents (greater than 20% by weight), unexpected problems arise during production: Once the hot stick compound has been filled into the stick mold, it must cool down in order to solidify and to be able to be removed from the mold as a stick. During production, however, the stick compound, which has become hygroscopic due to the glycerin, absorbs water from the ambient air, this having the result that the stick compound no longer hardens properly on cooling, but rather becomes softer and stickier and can no longer be removed (demolded) from the stick mold. It instead remains stuck to the mold.

In view thereof, it would be advantageous to be able to eliminate this disadvantage of the prior art and to develop a mineral oil-free and paraffin wax-free lipstick having a relatively high glycerin content (e.g., greater than about 20% by weight) which, during production, hardens on cooling and can then be removed from the stick mold without any problems.

### SUMMARY OF THE INVENTION

The present invention provides a cosmetic lipstick based on a glycerin-in-oil emulsion comprising
(a) Diisostearoyl Polyglyceryl-3 Dimer Dilinoleate,
(b) a second emulsifier selected from Polyglyceryl-4 Diisostearate/Polyhydroxystearate/Sebacate,
(c) sunflower wax
(d) carnauba wax.

The emulsion is free of mineral oil, paraffin wax, microcrystalline wax, shellac wax, polyethylene waxes, polyacrylates and crosslinked acrylate/C10-C30 alkyl acrylate polymers.

The present invention further provides a process for producing a lipstick based on a glycerin-in-oil emulsion, the oil phase whereof comprises
(a) Diisostearoyl Polyglyceryl-3 Dimer Dilinoleate,
(b) Polyglyceryl-4 Diisostearate/Polyhydroxystearate/Sebacate,
(c) sunflower wax and
(d) carnauba wax,
the preparation being free of mineral oil, paraffin wax, microcrystalline wax, shellac wax, polyethylene waxes, polyacrylates and crosslinked acrylate/C10-C30 alkyl acrylate polymers.

The process comprises first combining and homogenizing the glycerin phase and the oil phase at a temperature of in each case greater than/equal to 80° C.

Although WO 2021/239384 is known to those skilled in the art, this document was unable to point the way to the present invention.

Within the scope of the present disclosure, the phrases “according to the invention”, “preparation according to the invention”, etc. always relate to the preparations and processes according to the invention, i.e. also to preparations with which the process according to the invention is realized.

It is advantageous according to the invention if the preparation comprises glycerin in a concentration of from 21% to 30% by weight, based on the total weight of the preparation.

Furthermore, it is advantageous if the preparation comprises water in a concentration of less than 6% by weight, based on the total weight of the preparation.

Advantageous embodiments of the present invention are also characterized in that the preparation comprises Diisostearoyl Polyglyceryl-3 Dimer Dilinoleate in a concentration of from 1% to 4% by weight, based on the total weight of the preparation.

Moreover, it is advantageous in the context of the present invention if the preparation comprises Polyglyceryl-4 Diisostearate/Polyhydroxystearate/Sebacate in a concentration of from 0.5% to 2.5% by weight, based on the total weight of the preparation.

Furthermore, it is advantageous according to the invention if the preparation comprises sunflower wax in a concentration of from 1.0% to 10% by weight, based on the total weight of the preparation.

Advantageous embodiments in the context of the present invention are also characterized in that the preparation comprises carnauba wax in a concentration of from 0.3% to 2% by weight, based on the total weight of the preparation.

Moreover, it is advantageous in the context of the present invention if the preparation comprises behenyl behenate (INCI: Behenyl Behenate).

In such a case, it is preferred according to the invention if the preparation comprises behenyl behenate in a concentration of from 1.0% to 10% by weight, based on the total weight of the preparation.

It is also advantageous according to the invention if the preparation comprises beeswax (INCI: Cera Alba).

In such a case, it is preferred according to the invention if the preparation comprises beeswax in a concentration of from 1% to 10% by weight, based on the total weight of the preparation.

Moreover, the lipstick preparation may comprise one or more ingredients that are customary for such products, for example further lipophilic constituents, active ingredients such as coenzyme Q10, natural and/or synthetic isoflavonoids, flavonoids, creatine, creatinine, taurine, ß-alanine, panthenol, tocopheryl acetate, thiamidol, allantoin, vitamin E or vitamin E acetate, hyaluronic acid and/or salts thereof, licochalcone A and/or plant extracts.

The process according to the invention is advantageously characterized in that, after homogenization at a temperature of greater than/equal to 80° C., the preparation is cooled to a temperature of from 70° ° C. to 75° C. with stirring and then cast into a casting mold for sticks and cooled therein to room temperature at a cooling rate of 0.2-0.4° C./second.

Moreover, it is advantageous for the process if, after cooling to room temperature, the stick-shaped compound (stick crayon) is demolded and transferred into a storage container for stick-shaped preparations.

### DETAILED DESCRIPTION OF EXEMPLARY EMBODIMENTS

The examples which follow are intended to illustrate the present invention without limiting it. Unless otherwise indicated, all amounts, proportions and percentages are based on the weight and the total amount or on the total weight of the preparations.

| | | Exam- | Exam- | Exam- | Exam- |
|---|---|---|---|---|---|
| | INCI | ple A | ple B | ple C | ple D |
| Phase A | Behenyl Behenate | 5 | 4 | | 4 |
| | Prunus Amygdalus | 57.5 | 50.8 | 46.3 | 47.3 |
| | Dulcis Oil | | | | |
| | Cera Alba | | | 4 | |
| | Copernicia | 0.3 | 0.5 | 1.5 | 0.5 |
| | Cerifera Cera | | | | |
| | Ricinus Communis | 10 | 10 | 10 | 10 |
| | Seed Oil | | | | |
| | Helianthus Annuus | 1 | 2.5 | 2 | 2.5 |
| | Seed Cera | | | | |
| | Diisostearoyl | 1.4 | 1.4 | 1.4 | 1.4 |
| | Polyglyceryl-3 | | | | |
| | Dimer Dilinoleate | | | | |
| | Polyglyceryl-4 | 0.8 | 0.8 | 0.8 | 0.8 |
| | Diisostearate/ | | | | |
| | Polyhydroxystearate/ | | | | |
| | Sebacate | | | | |
| Phase B | Glycerin | 21 | 26 | 28 | 30 |
| | Aqua | 3 | 4 | 6 | 3.5 |
| | | 100 | 100 | 100 | 100 |

### Production of Solid Formulation:

Heat phase A to 90° C. with stirring
Heat phase B to 85° C. with stirring
Slowly add phase B to phase A, stir at 800-1200 rpm
Stir emulsion at 80° ° C. for a further 20 minutes
Then pour into the casting mold at 70-75° C.
Leave to cool at room temperature for 15 minutes. The cooling rate here is approx. 0.3° C./sec. Then leave to harden for a further 30 minutes at −18° C.
Recommended equipment: IKA stirrer with suitable dissolver

### Production of Creme Formulation:

Heat phase A to 90° C. with stirring
Heat phase B to 85° C. with stirring
Slowly add phase B to phase A, stir at 150 rpm
Homogenize hot emulsion
Then stir cold (50-70 rpm)
Homogenize emulsion again at 30° ° C.

What is claimed is:

1. A cosmetic lipstick based on a glycerin-in-oil emulsion, wherein the emulsion comprises
(a) Diisostearoyl Polyglyceryl-3 Dimer Dilinoleate,
(b) Polyglyceryl-4 Diisostearate/Polyhydroxystearate/Sebacate,
(c) sunflower wax, and
(d) carnauba wax,
the emulsion being free of mineral oil, paraffin wax, microcrystalline wax, shellac wax, polyethylene waxes, polyacrylates and crosslinked acrylate/C10-C30 alkyl acrylate polymers.

2. The lipstick of claim 1, wherein the emulsion comprises from 21% to 30% by weight of glycerin, based on a total weight of the emulsion.

3. The lipstick of claim 1, wherein the emulsion comprises less than 6% by weight of water, based on a total weight of the emulsion.

4. The lipstick of claim 1, wherein the emulsion comprises from 1% to 4% by weight of (a), based on a total weight of the emulsion.

5. The lipstick of claim 1, wherein the emulsion comprises from 0.5% to 2.5% by weight of (b), based on a total weight of the emulsion.

6. The lipstick of claim 1, wherein the emulsion comprises from 1.0% to 10% by weight of (c), based on a total weight of the emulsion.

7. The lipstick of claim 1, wherein the emulsion comprises from 0.3% to 2% by weight of (d), based on a total weight of the emulsion.

8. The lipstick of claim 1, wherein the emulsion further comprises behenyl behenate.

9. The lipstick of claim 8, wherein the emulsion comprises from 1% to 10% by weight of behenyl behenate, based on a total weight of the emulsion.

10. The lipstick of claim 1, wherein the emulsion further comprises beeswax.

11. The lipstick of claim 10, wherein the emulsion comprises from 1% to 10% by weight of beeswax, based on a total weight of the emulsion.

12. The lipstick of claim 1, wherein the emulsion comprises from 1% to 4% by weight of (a), from 0.5% to 2.5% by weight of (b), from 1.0% to 10% by weight of (c), and from 0.3% to 2% by weight of (d), based on a total weight of the emulsion.

13. The lipstick of claim 12, wherein the emulsion further comprises from 1.0% to 10% by weight of behenyl behenate, based on a total weight of the emulsion.

14. The lipstick of claim 12, wherein the emulsion further comprises from 1% to 10% by weight of beeswax, based on a total weight of the emulsion.

15. The lipstick of claim 13, wherein the emulsion further comprises from 1% to 10% by weight of beeswax, based on a total weight of the emulsion.

16. The lipstick of claim 1, wherein the emulsion further comprises one or both of Prunus amygdalus dulcis oil and Ricinus communis seed oil.

17. The lipstick of claim 1, wherein the emulsion further comprises Prunus amygdalus dulcis oil and Ricinus communis seed oil.

18. A process for producing the lipstick of claim 1, wherein the process comprises (i) first combining and homogenizing a glycerin phase and an oil phase which comprises components (a), (b), (c) and (d) at a temperature of in each case at least 80° C.

19. The process of claim 18, wherein the process further comprises (ii) cooling the emulsion obtained in (i) to a temperature of from 70° C. to 75° C. with stirring and then casting the emulsion into a casting mold for sticks and cooling the emulsion to room temperature at a cooling rate of 0.2-0.4° C./second.

20. The process of claim 19, wherein the process further comprises (iii) after cooling, demolding an obtained stick-shaped composition (stick crayon) and transferring the composition to a container for stick-shaped compositions.
